(19) United States
(12) Patent Application Publication
(10) Pub. No.: US 2025/0035614 A1
(43) Pub. Date: Jan. 30, 2025

(54) PLATFORM AND METHOD FOR ENGINEERING A HUMAN ORGANOID REPLICA FOR REPRODUCTIVE SCREENING

(71) Applicant: Gameto, Inc., New York, NY (US)

(72) Inventors: Christian KRAMME, New York, NY (US); Dina RADENKOVIC, New York, NY (US)

(21) Appl. No.: 18/424,348

(22) Filed: Jan. 26, 2024

Related U.S. Application Data
(63) Continuation of application No. 17/941,423, filed on Sep. 9, 2022, now abandoned.

(51) Int. Cl. G01N 33/50; C12N 5/071

(52) U.S. Cl. CPC G01N 33/5044; C12N 5/0682; C12N 2506/45; G01N 2800/364

(57) ABSTRACT

A platform and method for engineering a human organoid replica for reproductive screening. The platform including an engineered reproductive cell; a detector that measures a response by the engineered reproductive cell upon exposure of the engineered reproductive cell to a stimulus; a computing device in communication with the detector wherein the computing further at least a processor; and a memory communicatively connected to the processor, the memory containing instructions configuring the at least a processor to process stimulus data received from the detector; and compare data relating to the stimulus to a user profile.

### FIELD OF THE INVENTION

The present invention generally relates to the field of human organoids. In particular, the present invention is directed to a platform and method for engineering a human organoid replica for reproductive screening.

### BACKGROUND

Human organoids replicate the complexity and function of human organs. They serve as powerful tools to understand the molecular bases of developmental control and disease pathogenesis. However, there is a need for druggable human organoid models of the human reproductive system for reproductive screening.

### SUMMARY OF THE DISCLOSURE

In an aspect, a platform for engineering a human organoid replica for reproductive screening, the platform including an engineered reproductive cell; a detector that measures a response by the engineered reproductive cell upon exposure of the engineered reproductive cell to a stimulus; a computing device in communication with the detector wherein the computing further includes at least a processor; and a memory communicatively connected to the processor, the memory containing instructions configuring the at least a processor to process stimulus data received from the detector; and compare data relating to the stimulus to a user profile.

In another aspect, a method for engineering a human organoid replica for reproductive screening, the method including creating a user profile as a function of a reproductive cell relating to a user; receiving a plurality of human induced pluripotent stem cells; recapitulating, in vitro, the reproductive cell utilizing the plurality of human induced pluripotent stem cells; and generating, as a function of the recapitulated reproductive cell, a human organoid replica of an ovary.

These and other aspects and features of non-limiting embodiments of the present invention will become apparent to those skilled in the art upon review of the following description of specific non-limiting embodiments of the invention in conjunction with the accompanying drawings.

### BRIEF DESCRIPTION OF THE DRAWINGS

For the purpose of illustrating the invention, the drawings show aspects of one or more embodiments of the invention. However, it should be understood that the present invention is not limited to the precise arrangements and instrumentalities shown in the drawings, wherein:

FIG. 1 is an exemplary embodiment of a platform for engineering a human organoid replica for reproductive screening;

FIG. 2 an exemplary embodiment of a machine-learning module;

FIG. 3 an exemplary embodiment of neural network;

FIG. 4 is a diagram of an exemplary embodiment of a node of a neural network;

FIG. 5 is an exemplary flow diagram of a method for engineering a human organoid replica for reproductive screening;

FIG. 6 is an exemplary diagram of a human organoid replica applied as a disease model;

FIG. 7 is an exemplary diagram of a human organoid replica applied as a druggable disease model; and

FIG. 8 is a block diagram of a computing system that can be used to implement any one or more of the methodologies disclosed herein and any one or more portions thereof.

The drawings are not necessarily to scale and may be illustrated by phantom lines, diagrammatic representations and fragmentary views. In certain instances, details that are not necessary for an understanding of the embodiments or that render other details difficult to perceive may have been omitted.

### DETAILED DESCRIPTION

At a high level, aspects of the present disclosure are directed to systems and methods for transcription factor-directed differentiation methods to generate key human reproductive cell types from pluripotent stem cells. In an embodiment, these cell types can be utilized for high throughput 2D and 3D modeling of the human reproductive axis.

Aspects of the present disclosure can be used to streamlines and improves the costly and inefficient drug development process.

Exemplary embodiments illustrating aspects of the present disclosure are described below in the context of several specific examples.

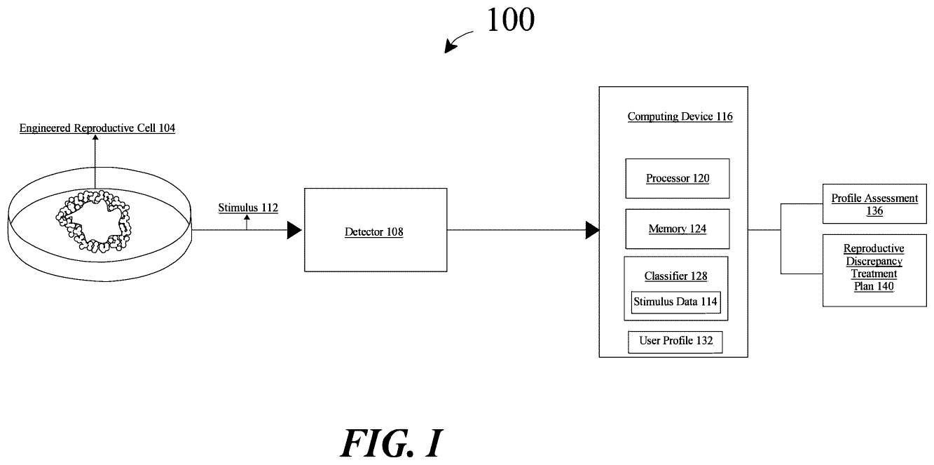

Referring now to FIG. 1, is an exemplary embodiment of a platform 100 for engineering a human organoid replica for reproductive screening. “Reproductive screening,” as used in this disclosure, is the testing of reproductive function and health. A “human organoid,” as used in this disclosure is a structure fabricated in vitro. “In vitro,” as used in this disclosure, is a process performed or taking place outside a living organism. As non-limiting examples, in a test tube, culture dish, and the like. Human organoid replica may be a two-dimensional or three-dimensional tissue like structure derived from stem cells that undergo self-organization and mimic the architecture and functionality of in vivo organs. “Stem cells,” as used in this disclosure, are cells with the potential to develop into many different types of cells in the body. “In vivo,” as used in this disclosure, is a process performed or taking place inside a living organism. A human organoid replica may be derived from pluripotent stem cells as describe further below, adult tissue stem cells, embryonic stem cells (hESCs), induced pluripotent stem cells (hIPSCs), and the like. In some embodiments, human organoid replicas of an ovary may be generated utilizing bioprinting. “Bioprinting,” as used in this disclosure is the utilization of 3D printing-like techniques to combine cells, growth factors, and/or biomaterials—to fabricate biomedical parts. Bioprinting a human organoid replica may include pre-bioprinting, bioprinting, and post-bioprinting. “Pre-bioprinting,” as used in this disclosure is the process of creating a model that a printer will later create and choosing the materials that will be used. This may include a biopsy of an organ such as an ovary. “Post-bioprinting,” as used in this disclosure, is the process of creating a stable structure from biological material. To maintain structure, both mechanical and chemical stimulations may be used. Stimulations may send signals to the cells to control the remodeling and growth of tissues in the human organoid replica. Methods of bioprinting may include direct printing, coaxial extrusion, indirect, laser, droplet and the like.

Still referring to FIG. 1, bioprinting may be performed without limitation by and/or using an additive manufacturing device. Additive manufacturing devices may include without limitation any device designed or configured to produce a component, organoid, human organoid, human organoid replica, product, or the like using an additive manufacturing process, in which material is deposited on the workpiece to be turned into the finished result. In some embodiments, an additive manufacturing process is a process in which material is added incrementally to a body of material in a series of two or more successive steps. The material may be added in the form of a stack of incremental layers; each layer may represent a cross-section of the object to be formed upon completion of the additive manufacturing process. Each cross-section may, as a non-limiting example be modeled on a computing device as a cross-section of graphical representation of the object to be formed; for instance, a computer aided design (CAD) tool may be used to receive or generate a three-dimensional model of the object to be formed, and a computerized process may derive from that model a series of cross-sectional layers that, when deposited during the additive manufacturing process, together will form the object. The steps performed by an additive manufacturing system to deposit each layer may be guided by a computer aided manufacturing (CAM) tool. In other embodiments, a series of layers are deposited in a substantially radial form, for instance by adding a succession of coatings to the workpiece. Similarly, the material may be added in volumetric increments other than layers, such as by depositing physical voxels in rectilinear or other forms. Additive manufacturing, as used in this disclosure, may specifically include manufacturing done at the atomic and nano level. Additive manufacturing also includes bodies of material that are a hybrid of other types of manufacturing processes, e.g. forging and additive manufacturing as described above. As an example, a forged body of material may have welded material deposited upon it which then comprises an additive manufactured body of material.

Deposition of material in additive manufacturing processes may be accomplished by any suitable means. Deposition may be accomplished using stereolithography, in which successive layers of polymer material are deposited and then caused to bind with previous layers using a curing process such as curing using ultraviolet light. Additive manufacturing processes may include “three-dimensional printing” processes that deposit successive layers of power and binder; the powder may include polymer or ceramic powder, and the binder may cause the powder to adhere, fuse, or otherwise join into a layer of material making up the body of material or product. Additive manufacturing may include metal three-dimensional printing techniques such as laser sintering including direct metal laser sintering (DMLS) or laser powder-bed fusion. Likewise, additive manufacturing may be accomplished by immersion in a solution that deposits layers of material on the body of material, by depositing and sintering materials having melting points such as metals, such as selective laser sintering, by applying fluid or paste-like materials in strips or sheets and then curing that material either by cooling, ultraviolet curing, and the like, any combination of the above methods, or any additional methods that involve depositing successive layers or other increments of material. Methods of additive manufacturing may include without limitation vat polymerization, material jetting, binder jetting, material extrusion, fuse deposition modeling, powder bed fusion, sheet lamination, and directed energy deposition. Methods of additive manufacturing may include adding material in increments of individual atoms, molecules, or other particles. An additive manufacturing process may use a single method of additive manufacturing, or combine two or more methods.

Additive manufacturing may include deposition of initial layers on a substrate. Substrate may include, without limitation, a support surface of an additive manufacturing device, or a removable item placed thereon. Substrate may include a base plate, which may be constructed of any suitable material; in some embodiments, where metal additive manufacturing is used, base plate may be constructed of metal, such as titanium. Base plate may be removable. One or more support features may also be used to support additively manufactured body of material during additive manufacture; for instance and without limitation, where a downward-facing surface of additively manufactured body of material is constructed having less than a threshold angle of steepness, support structures may be necessary to support the downward-facing surface; threshold angle May be, for instance 45 degrees. Support structures may be additively constructed, and may be supported on support surface and/or on upward-facing surfaces of additively manufactured body of material. Support structures may have any suitable form, including struts, buttresses, mesh, honeycomb or the like; persons skilled in the art, upon reviewing the entirety of this disclosure, will be aware of various forms that support structures may take consistently with the described methods and systems.

Additive manufacturing may alternatively create an element having biological elements, such as a organoid, human organoid, human organoid replica, by printing a support structure having a desired form, followed by deposition of living cells, tissues, or the like thereon. Support structure may be formed of any organic or inorganic material. As a non-limiting example, where organoid, human organoid, human organoid replica is intended to simulate the female reproductive system and/or any portion and/or organ thereof, a support structure may be used to replicate the form and/or structural attributes of pelvic bones, muscular and/or connective tissues surrounding and/or supporting such female reproductive system and/or portion and/or organ thereof, and/or additional portions of the female reproductive system the simulation of which is not necessary for a given application of methods and/or systems described herein. Construction of organoid, human organoid, human organoid replica may alternatively or additional involve use of additive manufacturing to construct a structural support, followed by a non-bioprinting application of biological materials, cell cultures or the like. Additive manufacturing and/or coating processes may alternatively or additionally be used to attach a growth medium, growth matrix, and/or layer of nutrients onto a substrate and/or support structure.

An additive manufacturing device may include an applicator or other additive device. For instance, an additive manufacturing device may include a printer head for a 3D printer. An additive manufacturing device may include an extruding device for extruding fluid or paste material, a sprayer or other applicator for bonding material, an applicator for powering, a sintering device such as a laser, or other such material.

An additive manufacturing device may include one or more robotic elements, including without limitation robot arms for moving, rotating, or otherwise positioning a workpiece, or for positioning a manufacturing tool, printer heads, or the like to work on workpiece. An additive manufacturing device may include one or more workpiece transport elements for moving a workpiece or finished part or component from one manufacturing stage to another; workpiece transport elements may include conveyors such as screw conveyors or conveyor belts, hoppers, rollers, or other items for moving an object from one place to another.

Still referring to FIG. 1, platform 100 includes an engineered reproductive cell 104. An “engineered reproductive cell,” as used in this disclosure, is a cell originating from a reproductive system engineered in vitro. Engineered reproductive cell 104 may include any type of cell, tissue, organ, and the like involved in a living organism's reproductive system. For example, engineered reproductive cell 104 may include an ovarian support cell such as a granulosa or theca cell, or a germline cell such as a gamete cell sourced from a sperm and/or egg. In yet another non-limiting example, engineered reproductive cell 104 may include an ovarian germ cell such as a non-gamete cell of the ovary and/or testis. In yet another non-limiting example, engineered reproductive cell 104 may include a mixture of one or more cells. “Engineering,” as used in this disclosure, is a process that alters and/or reproduces the genetic makeup of an organism. Engineered reproductive cell 104 may include one or more cells, tissue samples, a model of a reproductive system/organ/cell (e.g., an ovary, testicles, uterus, scrotum, etc.) as described further below. Engineering a reproductive cell 104 may include replicating genetic makeup and functionality of a received reproductive cell 104, organ, and/or system. For example, engineered reproductive cell 104 may be modeled after received reproductive cells, organs, and/or systems from a plurality of donors across various age groups. For example, replicating received ovarian cells may include analyzing and identifying discrepancies among the cells that may be engineered to generate and accurate replica in vitro representation. In an embodiment, engineered reproductive cell 104 may include a cell originating from a reproductive system in vivo.

Still referring to FIG. 1, in some embodiments, engineering may include altering the DNA makeup such as for example by changing a single base pair, deleting a region of DNA, adding a new segment of DNA, manipulating DNA, modifying DNA, recombining DNA and/or a nucleic acid, and the like. Engineering may include the design and construction of new biological entities such as with the use of laboratory technologies such as enzymes, genetic circuits, and cells or the redesign of existing biological systems. Engineering may include differentiating an engineered reproductive cell 104 to express one or more transcription factors, this process is referred to as transcription factor-directed cell differentiation throughout this disclosure. A “transcription factor,” as used in this disclosure is any protein that controls a rate of transcription. For example, a transcription factor may be selected from NR5A1 and a RUNX family protein. For instance, an engineered reproductive cell 104 may include an engineered granulosa cell configured to express and/or overexpress RUNX 1. An engineered reproductive cell 104 may express a particular protein and/or transcription factor if a level of the protein is detectable such as for example using a known protein assay. An engineered reproductive cell 104 may overexpress a particular protein and/or transcription factor if a particular protein and/or transcription factor level is detectable at a higher reference range. For example, an engineered reproductive cell 104 may overexpress a particular protein if the protein is detectable at a level that is 5% higher than the level of the protein expressed from an endogenous naturally occurring polynucleotide encoding the protein. Engineering may include engineering one or more polynucleotides of an engineered reproductive cell 104. An “engineered polynucleotide,” as used in this disclosure, is a nucleic acid that does not occur in nature. An engineered polynucleotide may include a recombinant nucleic acid. A “recombinant nucleic acid,” as used in this disclosure, is a molecule that is constructed by joining nucleic acids from two different organisms. For example, a recombinant nucleic acid may be created from a human and a mouse. An engineered polynucleotide may include a synthetic nucleic acid. A “synthetic nucleic acid,” as used in this disclosure, is a molecule that is amplified and/or chemically synthesized. For example, a synthetic nucleic acid may include a chemically modified and/or otherwise modified nucleic acid that can bind to one or more naturally occurring molecules. An engineered polynucleotide may include DNA (genomic DNA, cDNA, and/or any combination thereof), RNA, and/or a hybrid molecule. An engineered polynucleotide may include complementary DNA which may be synthesized from a single stranded RNA (messenger RNA (mRNA) or microRNA (miRNA)) such as for example using a catalyst such as but not limited to reverse transcriptase. In an embodiment, an engineered polynucleotide may include a promoter operably linked to an open reading frame. A “promoter,” as used in this disclosure, is a nucleotide sequence to which RNA polymerase binds to initiate transcription. A promoter may include an inducible promoter. An inducible promoter may be regulated in vitro by a stimulus 112 such as a chemical agent, temperature, or light. This may allow for temporal and/or spatial control of gene expression. For example, an inducible promoter may include but is not limited to an alcohol regulated promoter, a tetracycline operator sequence, a steroid regulated promoter, a human estrogen receptor, and the like. In an embodiment, engineering may include including altering of the cell's ability to express, overexpress and/or secrete a hormone including but not limited to hormones such as estrogen, progesterone, testosterone, DHEA and the like. An engineered reproductive cell 104 may include but is not limited to an engineered granulosa cell, an engineered lutein cell, and/or an engineered theca cell as described below in more detail. In an embodiment, an engineered reproductive cell 104 may be sourced from an oocyte, granulosa cell, cumulus oocyte complex, and similar cells originating in the ovary. An “oocyte,” as used in this disclosure, is a female gametocyte or germ cell involved in reproduction. In an embodiment, an engineered reproductive cell 104 may include an engineered granulosa cell. A “granulosa cell” is an estrogen-secreting cell of the epithelial lining of a graafian follicle and/or or its follicular precursor. Engineering may include any engineering process as described herein. For instance, and without limitation, a granulosa cell may be engineered to overexpress quantities of estradiol. “Estrogen” as used in this disclosure is a steroid hormone that promotes the development and/or maintenance of female sex characteristics. A “cumulus oocyte complex,” as used in this disclosure, is an oocyte containing one or more surrounding cumulus cells. A COC may contain an immature oocyte. A COC may contain a mature oocyte. A “mature oocyte” as used in this disclosure, is one or more mature reproductive cell 104s originating in the ovaries. In some embodiments, engineered reproductive cell 104 may include but is not limited to an engineered cell and/or any combination thereof including oogonia cells, oogonia-like pluripotent stem cells, polynucleotides, primordial germ cells, and primordial germ cell-like pluripotent stem cells. For example, engineered reproductive cell 104 may include a pluripotent stem cell (PSC) incorporating: an engineered polynucleotide including an open reading frame encoding a protein selected from Zinc Finger Protein 281 (ZNF281), LIM Homeobox 8 (LHX8), and Spermatogenesis and Oogenesis Specific Basic Helix-Loop-Helix 1 (SOHLH1). In yet another non-limiting example, engineered reproductive cell 104 may include a pluripotent stem cell (PSC) incorporating an engineered polynucleotide including an open reading frame encoding a protein selected from Distal-Less Homeobox 5 (DLX5), Hematopoietically-expressed homeobox protein (HHEX), and Folliculogenesis Specific BHLH Transcription Factor (FIGLA). In yet another non-limiting example, engineered reproductive cell 104 may include a pluripotent stem cell (PSC) incorporating an engineered polynucleotide including an open reading frame encoding a protein selected from nuclear receptor subfamily 5 group A member 1 (NR5A1) and a Runt-related transcription factor (RUNX) family protein.

Still referring to FIG. 1, in an embodiment, a reproductive cell 104 may be engineered using Clustered Regularly Interspaced Short Palindromic Repeats (CRISPR) technology. “CRISPR” is programmable technology that targets specific stretches of genetic code to edit DNA at precise locations. CRISPR technology may include CRISPR-CAS 9. Cas9 (or “CRISPR-associated protein 9”) is an enzyme that uses CRISPR sequences as a guide to recognize and cleave specific strands of DNA that are complementary to the CRISPR sequence. Cas9 enzymes together with CRISPR sequences form the basis of a technology known as CRISPR-Cas9 that can be used to edit genes within organisms. CRISPR technology may include Class 1 CRISPR systems including type I (cas3), type III (cas10), and type IV and 12 subtypes. CRISPR technology may include Class 2 CRISPR systems including type II (cas9), type V (cas12), type VI (cas13), and 9 subtypes. In some embodiments, CRISPR technology may involve CRISPR-Cas design tools which are computer software platform 100s and bioinformatics tools used to facilitate the design of guide RNAs (gRNAs) for use with the CRISPR/Cas gene editing system. For example, CRISPR-Cas design tools may include: CRISPRon, CRISPRoff, Invitrogen TrueDesign Genome Editor, Breaking-Cas, Cas-OFFinder, CASTING, CRISPy, CCTop, CHOPCHOP, CRISPOR, sgRNA Designer, Synthego Design Tool, and the like. CRISPR technology may also be used as a diagnostic tool. For example, CRISPR-based diagnostics may be coupled to enzymatic processes, such as SHERLOCK-based Profiling of IN vitro Transcription (SPRINT). SPRINT can be used to detect a variety of substances, such as metabolites in patient samples or contaminants in environmental samples, with high throughput or with portable point-of-care devices.

Still referring to FIG. 1, in some embodiments, engineered reproductive cell 104 may be derived from a pluripotent stem cell. “Pluripotent stem cells,” as used in this disclosure, are cells that are able to self-renew by dividing and developing into the three primary groups of cells that make up a human body, including ectoderm, giving rise to the skin and nervous system; endoderm, forming the gastrointestinal and respiratory tracts, endocrine glands, liver, and pancreas; and mesoderm, forming bone, cartilage, most of the circulatory system, muscles, connective tissue, and more. Pluripotent stem cells may be able to make cells from all three of these basic body layers, so they can potentially produce any cell or tissue the body needs to repair itself. Pluripotent stem cells may include induced pluripotent stem cells (iPSCs), which are derived from skin or blood cells that have been reprogrammed back into an embryonic-like pluripotent state that may enable the development of an unlimited source of any type of human cell needed for therapeutic purposes. For example, iPSC can be prodded into becoming beta islet cells to treat diabetes, blood cells to create new blood free of cancer cells for a leukemia patient, or neurons to treat neurological disorders. Induced pluripotent cells may be derived from embryos, embryonic stem cells made by somatic cell nuclear transfer (ntESCs) and/or an embryonic stem cell from an unfertilized egg. In an embodiment, a pluripotent cell may include a human pluripotent cell. In an embodiment, a pluripotent cell may include an embryonic stem cell, such as a human embryonic stem cell. An “embryonic stem cell,” as used in this disclosure, is a pluripotent stem cell made using embryos or eggs. An embryonic stem cell may include but is not limited to a true embryonic stem cell, a nuclear transfer embryonic stem cell, and/or a parthenogenetic embryonic stem cell. In an embodiment, a pluripotent stem cell may include an induced pluripotent stem cell such as a human induced pluripotent stem cell. A human induced pluripotent stem cell may be derived from skin or blood cells that may be engineered back into an embryonic-like pluripotent state that enables the development of an unlimited source of any type of human cell. In some embodiments, engineered reproductive cell 104 may include an engineered a theca cell. A “theca cell,” as used in this disclosure, is one or more endocrine cells associated with ovarian follicles that produce androgens. Engineering may include any engineering process as described herein. In some embodiments, the engineered cell may be a lutein cell. A “lutein cell,” as used in this disclosure, is a cell of the corpus luteum. Engineering may include any engineering process as described herein.

Still referring to FIG. 1, engineering a reproductive cell 104 may include utilizing in silico target discovery. “In silico,” as used in this disclosure, are biological models developed on a computer to model a pharmacologic or physiologic process using computer simulation. In silico discovery of potential biological targets for chemical compounds may offer an alternative avenue for the exploration of ligand-target interactions and biochemical mechanisms, as well as for investigation of drug repurposing. A “biological target” as used in this disclosure, is anything within a living organism to which some other entity (like an endogenous ligand or a drug) is directed and/or binds, resulting in a change in its behavior or function. Examples of common classes of biological targets may include proteins and nucleic acids. For example, in silico discovery techniques may be used to model a reproductive cell 104 for drug and disease testing. In some embodiments, a biological target may be a native protein in the body whose activity is modified by a drug resulting in a specific effect, which may be a desirable therapeutic effect or an unwanted adverse effect. In some embodiments, in silico discovery may be used to model a reproductive cell 104 to discover transcriptomic and proteomic signatures to replicate in human organoid replica. In silico target discovery may include computational target fishing mines biologically annotated chemical databases and then maps compound structures into chemogenomic space to predict the biological targets. Applications in computational target fishing may include chemical similarity searching, data mining/machine learning, panel docking, and the bioactivity spectral analysis for target identification. In some embodiments, engineering a reproductive cell 104 may include utilizing microfluidics. “Microfluidics,” as used in this disclosure, is the behavior, precise control, and manipulation of fluids that are geometrically constrained to a small scale (typically sub-millimeter) at which surface forces dominate volumetric forces. For example, microfluidics for cell biology may be a mini cell culture system where a single cell or a few cells are seeded into a device with input and output channels. These cells may be exposed to dynamic fluid flow, accompanied by live imaging. Microfluidics systems may be used in capillary electrophoresis, isoelectric focusing, immunoassays, flow cytometry, optimization of protein drugs production, sample injection in mass spectrometry, PCR amplification, DNA analysis, separation and manipulation of cells, and cell patterning of a received ovarian cell/and or engineered reproductive cell 104.

Still referring to FIG. 1, platform 100 includes a detector 108 that measures a response by the engineered reproductive cell 104 upon exposure of the engineered reproductive cell 104 to a stimulus 112. A “stimulus,” as used in thus disclosure, is an element that triggers a physical or functional change. A “detector,” as used in this disclosure is a sensor capable measuring a cell response. For example, detector 108 may include detectors that utilize liquid chromatography, mass spectrometer, assays, flow cytometer, and the like. In some embodiments, stimulus 112 may relate to drug metabolism. “Drug metabolism,” as used in this disclosure, is the metabolic breakdown of drugs in cells. The metabolism of pharmaceutical drugs may be an important aspect of pharmacology and medicine. For example, the rate of metabolism determines the duration and intensity of a drug's pharmacologic action. In some embodiments, human organoid replica may be used for preclinical studies for all products in woman's health. By testing drug products on human organoid replica, the success rate of a product may be predicted, tested and/or fine-tuned before clinical and clinical trials. For example, engineered reproductive cell 104 replicating a lutein cell producing low estrogen levels may be stimulate with drugs products configured to increase estrogen production. Detector 108 may then be used to measure the response of engineered reproductive cell 104 to stimulus 112. Data collected from detector 108 may be stored by a computing device 116 and processed as described further below. In some embodiments, stimulus 112 may relate to a toxicity screening. A “toxicity screening,” as used in this disclosure, is a test to identify an element that has a deleterious effect on cell functions and development. An element may include a drug or common molecule. For example an element may be drugs deleterious to the health of ovarian cells, such as cyclophosphamide, cisplatin and doxorubicin, which may cause premature ovarian insufficiency by inducing death and/or accelerated activation of primordial follicles and increased atresia of growing follicles. In some embodiments, human organoid replica may be used to screen drugs entering clinical trials to ensure the drugs are not toxic to the ovaries and thus allow more woman to safely enroll in drug related trials.

Still referring to FIG. 1, in some embodiments, stimulus 112 may relate to a disease model. A “disease model,” as used in this disclosure, is a cell displaying a pathological process that is observed in at least an actual human disease. For example, human organoid replica may model an ovary experiencing endometriosis. There may be few to no targeted treatments for endometriosis currently and despite interest from pharmaceutical companies, there may be few to no effective models for endometriosis drug development. Human organoid replica may provide an accurate disease model of endometriosis for drug/treatment development, medicinal studies, and the like. In some embodiments, stimulus 112 may relate to an epigenetic model. An “epigenetic model,” as used in this disclosure, is an organoid used to study heritable phenotype changes that do not involve alterations in the DNA sequence. Epigenetic changes in human organoid replica may include DNA Methylation, Histone modification, Non-coding RNA, and similar changes. For example, epigenetic changes in human organoid replica may be analyzed to determine the effects of anti-aging interventions. The effects of anti-aging interventions may be measured in a shorter time frame than in vivo organs. As a disease model, therapeutics for ovarian reserve preservation and oocyte improvement may tested. For example, peptide AMH mimetics as AMHR2 agonists to fine-tune follicle recruitment and prevent premature ovarian failure and early-onset menopause. Peptide mimics of AMH can fine-tune AMHR2 signaling and prevent over-recruitment of primary follicles, thus preserving the ovarian reserve.

Still referring to FIG. 1, platform 100 includes a computing device 116. Computing device 116 may include any computing device as described in this disclosure, including without limitation a microcontroller, microprocessor 120, digital signal processor 120 (DSP) and/or system on a chip (SoC) as described in this disclosure. Computing device 116 includes a processor 120 and a memory 124 communicatively connected to the processor 120, wherein memory 124 contains instructions configuring processor 120 to process and compare data related to a stimulus response. As used in this disclosure, “communicatively connected” means connected by way of a connection, attachment, or linkage between two or more relata which allows for reception and/or transmittance of information therebetween. For example, and without limitation, this connection may be wired or wireless, direct, or indirect, and between two or more components, circuits, devices, systems, and the like, which allows for reception and/or transmittance of data and/or signal(s) therebetween. Data and/or signals therebetween may include, without limitation, electrical, electromagnetic, magnetic, video, audio, radio, and microwave data and/or signals, combinations thereof, and the like, among others. A communicative connection may be achieved, for example and without limitation, through wired or wireless electronic, digital, or analog, communication, either directly or by way of one or more intervening devices or components. Further, communicative connection may include electrically coupling or connecting at least an output of one device, component, or circuit to at least an input of another device, component, or circuit. For example, and without limitation, via a bus or other facility for intercommunication between elements of a computing device. Communicative connecting may also include indirect connections via, for example and without limitation, wireless connection, radio communication, low power wide area network, optical communication, magnetic, capacitive, or optical coupling, and the like. In some instances, the terminology “communicatively coupled” may be used in place of communicatively connected in this disclosure. Computing device 116 may include, be included in, and/or communicate with a mobile device such as a mobile telephone or smartphone. Computing device 116 may include a single computing device 116 operating independently or may include two or more computing device 116 operating in concert, in parallel, sequentially or the like; two or more computing devices 116s may be included together in a single computing device 116 or in two or more computing devices 116. Computing device 116 may interface or communicate with one or more additional devices as described below in further detail via a network interface device. Network interface device may be utilized for connecting computing device 116 to one or more of a variety of networks, and one or more devices. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices 116, and any combinations thereof. A network may employ a wired and/or a wireless mode of communication. In general, any network topology may be used. Information (e.g., data, software etc.) may be communicated to and/or from a computer and/or a computing device 116. Computing device 116 may include but is not limited to, for example, a computing device 116 or cluster of computing devices 116 in a first location and a second computing device 116 or cluster of computing devices 116 in a second location. Computing device 116 may include one or more computing devices 116 dedicated to data storage, security, distribution of traffic for load balancing, and the like. Computing device 116 may distribute one or more computing tasks as described below across a plurality of computing devices 116 of computing device 116, which may operate in parallel, in series, redundantly, or in any other manner used for distribution of tasks or memory 124 between computing devices 116. Computing device 116 may be implemented using a “shared nothing” architecture in which data is cached at the worker, in an embodiment, this may enable scalability of system 100 and/or computing device 116.

With continued reference to FIG. 1, computing device 116 may be designed and/or configured to perform any method, method step, or sequence of method steps in any embodiment described in this disclosure, in any order and with any degree of repetition. For instance, computing device 116 may be configured to perform a single step or sequence repeatedly until a desired or commanded outcome is achieved; repetition of a step or a sequence of steps may be performed iteratively and/or recursively using outputs of previous repetitions as inputs to subsequent repetitions, aggregating inputs and/or outputs of repetitions to produce an aggregate result, reduction or decrement of one or more variables such as global variables, and/or division of a larger processing task into a set of iteratively addressed smaller processing tasks. Computing device 116 may perform any step or sequence of steps as described in this disclosure in parallel, such as simultaneously and/or substantially simultaneously performing a step two or more times using two or more parallel threads, processor 120 cores, or the like; division of tasks between parallel threads and/or processes may be performed according to any protocol suitable for division of tasks between iterations. Persons skilled in the art, upon reviewing the entirety of this disclosure, will be aware of various ways in which steps, sequences of steps, processing tasks, and/or data may be subdivided, shared, or otherwise dealt with using iteration, recursion, and/or parallel processing.

Still referring to FIG. 1, computing device 116 is configured to process stimulus data 114. Processing data may include analyzing engineered reproductive cell 104 response to stimulus 112 to determine cell health changes, metabolic change, expression changes, and/or genome changes. “Cell health changes,” as used in this disclosure, are changes in the wellness and function of a cell. Determining cell health changes may include utilizing cell viability assays, cytotoxicity assays, apoptosis assays, autophagic assays, autophagic flux, ADME assays, and the like. “Cell viability assays,” as used in this disclosure, are assays created to determine the ability of cells to maintain a state of survival. This may include the ability of cells to recover a state of survival. Determining metabolic changes may include utilizing dinucleotide assays, energy metabolite assays, oxidative stress assays, and the like. Determining expression changes may include utilizing RNA isolation, dye-based/probe-based qPCR and RT-qPCR reagents, promoter analysis receptor assays, and the like. Determining genome changes may include utilizing STR profiling for cell line identity, NGS/Directed Sequencing/SNP, genotyping, epigenetic profile, and the like. In some embodiments, computing device 116 may be configured to process data using a machine algorithm such as a classifier 128. A “classifier,” as used in this disclosure is a machine-learning model, such as a mathematical model, neural net, or program generated by a machine learning algorithm known as a “classification algorithm,” as described in further detail below, that sorts inputs into categories or bins of data, outputting the categories or bins of data and/or labels associated therewith. classifier 128 may be configured to output at least a datum that labels or otherwise identifies a set of data that are clustered together, found to be close under a distance metric as described below, or the like. Computing device 116 and/or another device may generate classifier 128 using a classification algorithm, defined as a processes whereby computing device 116 derives a classifier from training data. Classification may be performed using, without limitation, linear classifiers such as without limitation logistic regression and/or naive Bayes classifiers, nearest neighbor classifiers such as k-nearest neighbors classifiers, support vector machines, least squares support vector machines, fisher's linear discriminant, quadratic classifiers, decision trees, boosted trees, random forest classifiers, learning vector quantization, and/or neural network-based classifiers.

Still referring to FIG. 1, classifier 128 may be used for image-based profile assessment 136 of engineered reproductive cell 104 response to stimuli. A “profile assessment,” as used in this disclosure is a cell evaluation measuring a large number of features in a cell. For example, area, shape, intensity, texture, and other aspects. A profile assessment may utilize assays as described above. Computing device may be configured to analyze microscopy image formats. Additionally computing device may be configured to perform microscopy image processing as part of image-based profile assessment 136. “Microscopy image processing,” as used in this disclosure, is the use of digital image processing techniques to process, analyze and present images obtained from a microscope. An image-based profiling assessment may include assays assessing single-cell phenotypes may be used to explore mechanisms of action, target efficacy and toxicity of small molecules. For example, classifier 128 may receive culture images from detector 108 wherein classifier 128 is configured to generate a phenotypic profiling of reproductive cell 104. Computing device 116 may train classifier 128 using training data including, images, labels and descriptions of cells or cell lines representing a plurality of cell morphology in response to a plurality of different stimuli. Training data may contain images of cell samples of various age groups. For example, training data may contain fetal cell images in response acitretin. Training data may contain image of cells in response to specific stimulus 112 dosing. For example, images of cells exposed to 10 mg of cetirizine. In some embodiments, classifier 128 may be configured to perform scoring methods, such as total oocyte scoring as described in U.S. Nonprovisional application Ser. No. 17/846,725, filed on Jun. 22, 2022, and entitled “AN APPARATUS AND METHOD FOR INDUCING HUMAN OOCYTE MATURATION IN VITRO,” the entirety of which is incorporated herein by reference.

Still referring to FIG. 1, computing device 116 may be configured to generate classifier 128 using a Naïve Bayes classification algorithm. Naïve Bayes classification algorithm generates classifiers by assigning class labels to problem instances, represented as vectors of element values. Class labels are drawn from a finite set. Naïve Bayes classification algorithm may include generating a family of algorithms that assume that the value of a particular element is independent of the value of any other element, given a class variable. Naïve Bayes classification algorithm may be based on Bayes Theorem expressed as P(A/B)=P(B/A)=P(A)=P(B), where P(A/B) is the probability of hypothesis A given data B also known as posterior probability; P(B/A) is the probability of data B given that the hypothesis A was true; P(A) is the probability of hypothesis A being true regardless of data also known as prior probability of A; and P(B) is the probability of the data regardless of the hypothesis. A naïve Bayes algorithm may be generated by first transforming training data into a frequency table. Computing device 116 may then calculate a likelihood table by calculating probabilities of different data entries and classification labels. Computing device 116 may utilize a naïve Bayes equation to calculate a posterior probability for each class. A class containing the highest posterior probability is the outcome of prediction. Naïve Bayes classification algorithm may include a gaussian model that follows a normal distribution. Naïve Bayes classification algorithm may include a multinomial model that is used for discrete counts. Naïve Bayes classification algorithm may include a Bernoulli model that may be utilized when vectors are binary.

With continued reference to FIG. 1, computing device 116 may be configured to generate classifier 128 using a K-nearest neighbors (KNN) algorithm. A “K-nearest neighbors algorithm” as used in this disclosure, includes a classification method that utilizes feature similarity to analyze how closely out-of-sample-features resemble training data to classify input data to one or more clusters and/or categories of features as represented in training data; this may be performed by representing both training data and input data in vector forms, and using one or more measures of vector similarity to identify classifications within training data, and to determine a classification of input data. K-nearest neighbors algorithm may include specifying a K-value, or a number directing the classifier to select the k most similar entries training data to a given sample, determining the most common classifier of the entries in the database, and classifying the known sample; this may be performed recursively and/or iteratively to generate a classifier that may be used to classify input data as further samples. For instance, an initial set of samples may be performed to cover an initial heuristic and/or “first guess” at an output and/or relationship, which may be seeded, without limitation, using expert input received according to any process as described herein. As a non-limiting example, an initial heuristic may include a ranking of associations between inputs and elements of training data. Heuristic may include selecting some number of highest-ranking associations and/or training data elements.

With continued reference to FIG. 1, generating k-nearest neighbors algorithm may generate a first vector output containing a data entry cluster, generating a second vector output containing an input data, and calculate the distance between the first vector output and the second vector output using any suitable norm such as cosine similarity, Euclidean distance measurement, or the like. Each vector output may be represented, without limitation, as an n-tuple of values, where n is at least two values. Each value of n-tuple of values may represent a measurement or other quantitative value associated with a given category of data, or attribute, examples of which are provided in further detail below; a vector may be represented, without limitation, in n-dimensional space using an axis per category of value represented in n-tuple of values, such that a vector has a geometric direction characterizing the relative quantities of attributes in the n-tuple as compared to each other. Two vectors may be considered equivalent where their directions, and/or the relative quantities of values within each vector as compared to each other, are the same; thus, as a non-limiting example, a vector represented as [5, 10, 15] may be treated as equivalent, for purposes of this disclosure, as a vector represented as [1, 2, 3]. Vectors may be more similar where their directions are more similar, and more different where their directions are more divergent; however, vector similarity may alternatively or additionally be determined using averages of similarities between like attributes, or any other measure of similarity suitable for any n-tuple of values, or aggregation of numerical similarity measures for the purposes of loss functions as described in further detail below. Any vectors as described herein may be scaled, such that each vector represents each attribute along an equivalent scale of values. Each vector may be “normalized,” or divided by a “length” attribute, such as a length attribute l as derived using a Pythagorean norm: l=\sqrt{Σᵢ₌₀ⁿaᵢ²}, where aᵢ is attribute number i of the vector. Scaling and/or normalization may function to make vector comparison independent of absolute quantities of attributes, while preserving any dependency on similarity of attributes; this may, for instance, be advantageous where cases represented in training data are represented by different quantities of samples, which may result in proportionally equivalent vectors with divergent values.

Still referring to FIG. 1, platform 100 includes a computing device 116 configured to compare stimulus data 114 to a user profile 132. “Stimulus data,” as used in this disclosure, is cell response to a stimulus received from a detector. A “user profile,” as used in this disclosure is a data structure containing analytical data relating to a user. The user profile 132 may contain biological information related to the user such as age, race, family genetics, reproductive cell 104s, and the like. For example, a user profile 132 may include an ovarian disease such as polycystic ovary syndrome being a familial condition related to the user and the likely of the user experiencing the condition. A “user,” as used in this disclosure is a person. Computing device may generate a classifier 128 as defined above to output a reproductive discrepancy treatment plan 140 for a user. A “reproductive discrepancy treatment plan,” as used in this disclosure is a treatment plan aimed to mitigate a reproductive discrepancy. A “treatment plan” as used in this disclosure, is a detailed proposal for the treatment of a condition. In some embodiments, a reproductive discrepancy treatment plan 140 may be a detailed plan with information about a user's disease, the goal of treatment, the treatment options for the disease and possible side effects, and the expected length of treatment. For example, a reproductive discrepancy treatment plan 140 may be a treatment plan 140 with the goal of countering infertility, hirsutism, acne and/or obesity issues caused by polycystic ovary syndrome. Training data for the classifier 128 may include correlations between user profile 132, stimulus data 114, and data retrieved from a treatment database. A “treatment database,” as used in this disclosure is a data structure containing a plurality of information relating to reproductive cell 104 health, the reproductive system, historical methods of reproductive discrepancy treatment, experimental methods of reproductive discrepancy treatment, side effects associated with treatment plans 140, side effects of medications, and treatment plans 140 associated with certain qualities of a user (e.g., genetics, medical history) and the like. All databases described throughput this disclosure may be communicatively connected to computing device 116 and may be implemented, without limitation, as a relational database, a key-value retrieval database such as a NOSQL database, or any other format or structure for use as a database that a person skilled in the art would recognize as suitable upon review of the entirety of this disclosure. Database may alternatively or additionally be implemented using a distributed data storage protocol and/or data structure, such as a distributed hash table or the like. Database may include a plurality of data entries and/or records as described above. Data entries in a database may be flagged with or linked to one or more additional elements of information, which may be reflected in data entry cells and/or in linked tables such as tables related by one or more indices in a relational database. Persons skilled in the art, upon reviewing the entirety of this disclosure, will be aware of various ways in which data entries in a database may store, retrieve, organize, and/or reflect data and/or records as used herein, as well as categories and/or populations of data consistently with this disclosure. Comparison may be performed using any suitable algorithm, including without limitation a classifier, which may include any type of classifier described herein; classifier may be trained using training examples correlating user profiles to stimulus data, which training examples may be entered by users, collected using previous iterations of methods described herein, or the like.

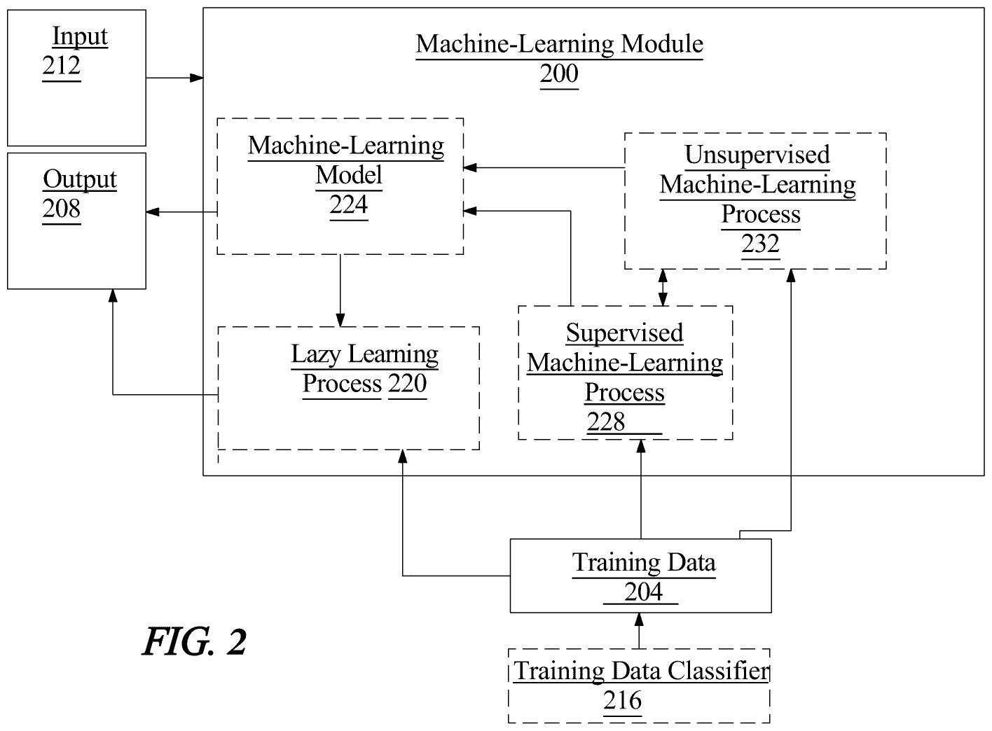

Referring now to FIG. 2, an exemplary embodiment of a machine-learning module 200 that may perform one or more machine-learning processes as described in this disclosure is illustrated. Machine-learning module may perform determinations, classification, and/or analysis steps, methods, processes, or the like as described in this disclosure using machine learning processes. A “machine learning process,” as used in this disclosure, is a process that automatedly uses training data 204 to generate an algorithm that will be performed by a computing device/module to produce outputs 208 given data provided as inputs 212; this is in contrast to a non-machine learning software program where the commands to be executed are determined in advance by a user and written in a programming language.

Still referring to FIG. 2, “training data,” as used herein, is data containing correlations that a machine-learning process may use to model relationships between two or more categories of data elements. For instance, and without limitation, training data 204 may include a plurality of data entries, each entry representing a set of data elements that were recorded, received, and/or generated together; data elements may be correlated by shared existence in a given data entry, by proximity in a given data entry, or the like. Multiple data entries in training data 204 may evince one or more trends in correlations between categories of data elements; for instance, and without limitation, a higher value of a first data element belonging to a first category of data element may tend to correlate to a higher value of a second data element belonging to a second category of data element, indicating a possible proportional or other mathematical relationship linking values belonging to the two categories. Multiple categories of data elements may be related in training data 204 according to various correlations; correlations may indicate causative and/or predictive links between categories of data elements, which may be modeled as relationships such as mathematical relationships by machine-learning processes as described in further detail below. Training data 204 may be formatted and/or organized by categories of data elements, for instance by associating data elements with one or more descriptors corresponding to categories of data elements. As a non-limiting example, training data 204 may include data entered in standardized forms by persons or processes, such that entry of a given data element in a given field in a form may be mapped to one or more descriptors of categories. Elements in training data 204 may be linked to descriptors of categories by tags, tokens, or other data elements; for instance, and without limitation, training data 204 may be provided in fixed-length formats, formats linking positions of data to categories such as comma-separated value (CSV) formats and/or self-describing formats such as extensible markup language (XML), JavaScript Object Notation (JSON), or the like, enabling processes or devices to detect categories of data.

Alternatively or additionally, and continuing to refer to FIG. 2, training data 204 may include one or more elements that are not categorized; that is, training data 204 may not be formatted or contain descriptors for some elements of data. Machine-learning algorithms and/or other processes may sort training data 204 according to one or more categorizations using, for instance, natural language processing algorithms, tokenization, detection of correlated values in raw data and the like; categories may be generated using correlation and/or other processing algorithms. As a non-limiting example, in a corpus of text, phrases making up a number “n” of compound words, such as nouns modified by other nouns, may be identified according to a statistically significant prevalence of n-grams containing such words in a particular order; such an n-gram may be categorized as an element of language such as a “word” to be tracked similarly to single words, generating a new category as a result of statistical analysis. Similarly, in a data entry including some textual data, a person's name may be identified by reference to a list, dictionary, or other compendium of terms, permitting ad-hoc categorization by machine-learning algorithms, and/or automated association of data in the data entry with descriptors or into a given format. The ability to categorize data entries automatedly may enable the same training data 204 to be made applicable for two or more distinct machine-learning algorithms as described in further detail below. Training data 204 used by machine-learning module 200 may correlate any input data as described in this disclosure to any output data as described in this disclosure.

Further referring to FIG. 2, training data may be filtered, sorted, and/or selected using one or more supervised and/or unsupervised machine-learning processes and/or models as described in further detail below; such models may include without limitation a training data classifier 216. Training data classifier 216 may include a “classifier,” which as used in this disclosure is a machine-learning model as defined below, such as a mathematical model, neural net, or program generated by a machine learning algorithm known as a “classification algorithm,” as described in further detail below, that sorts inputs into categories or bins of data, outputting the categories or bins of data and/or labels associated therewith. A classifier may be configured to output at least a datum that labels or otherwise identifies a set of data that are clustered together, found to be close under a distance metric as described below, or the like. Machine-learning module 200 may generate a classifier using a classification algorithm, defined as a processes whereby a computing device and/or any module and/or component operating thereon derives a classifier from training data 204. Classification may be performed using, without limitation, linear classifiers such as without limitation logistic regression and/or naïve Bayes classifiers, nearest neighbor classifiers such as k-nearest neighbors classifiers, support vector machines, least squares support vector machines, fisher's linear discriminant, quadratic classifiers, decision trees, boosted trees, random forest classifiers, learning vector quantization, and/or neural network-based classifiers.

Still referring to FIG. 2, machine-learning module 200 may be configured to perform a lazy-learning process 220 and/or protocol, which may alternatively be referred to as a “lazy loading” or “call-when-needed” process and/or protocol, may be a process whereby machine learning is conducted upon receipt of an input to be converted to an output, by combining the input and training set to derive the algorithm to be used to produce the output on demand. For instance, an initial set of simulations may be performed to cover an initial heuristic and/or “first guess” at an output and/or relationship. As a non-limiting example, an initial heuristic may include a ranking of associations between inputs and elements of training data 204. Heuristic may include selecting some number of highest-ranking associations and/or training data 204 elements. Lazy learning may implement any suitable lazy learning algorithm, including without limitation a K-nearest neighbors algorithm, a lazy naïve Bayes algorithm, or the like; persons skilled in the art, upon reviewing the entirety of this disclosure, will be aware of various lazy-learning algorithms that may be applied to generate outputs as described in this disclosure, including without limitation lazy learning applications of machine-learning algorithms as described in further detail below.

Alternatively or additionally, and with continued reference to FIG. 2, machine-learning processes as described in this disclosure may be used to generate machine-learning models 224. A “machine-learning model,” as used in this disclosure, is a mathematical and/or algorithmic representation of a relationship between inputs and outputs, as generated using any machine-learning process including without limitation any process as described above and stored in memory; an input is submitted to a machine-learning model 224 once created, which generates an output based on the relationship that was derived. For instance, and without limitation, a linear regression model, generated using a linear regression algorithm, may compute a linear combination of input data using coefficients derived during machine-learning processes to calculate an output datum. As a further non-limiting example, a machine-learning model 224 may be generated by creating an artificial neural network, such as a convolutional neural network comprising an input layer of nodes, one or more intermediate layers, and an output layer of nodes. Connections between nodes may be created via the process of “training” the network, in which elements from a training data 204 set are applied to the input nodes, a suitable training algorithm (such as Levenberg-Marquardt, conjugate gradient, simulated annealing, or other algorithms) is then used to adjust the connections and weights between nodes in adjacent layers of the neural network to produce the desired values at the output nodes. This process is sometimes referred to as deep learning.

Still referring to FIG. 2, machine-learning algorithms may include at least a supervised machine-learning process 228. At least a supervised machine-learning process 228, as defined herein, include algorithms that receive a training set relating a number of inputs to a number of outputs, and seek to find one or more mathematical relations relating inputs to outputs, where each of the one or more mathematical relations is optimal according to some criterion specified to the algorithm using some scoring function. For instance, a supervised learning algorithm may include inputs and outputs, as described above, and a scoring function representing a desired form of relationship to be detected between inputs and outputs; scoring function may, for instance, seek to maximize the probability that a given input and/or combination of elements inputs is associated with a given output to minimize the probability that a given input is not associated with a given output. Scoring function may be expressed as a risk function representing an “expected loss” of an algorithm relating inputs to outputs, where loss is computed as an error function representing a degree to which a prediction generated by the relation is incorrect when compared to a given input-output pair provided in training data 204. Persons skilled in the art, upon reviewing the entirety of this disclosure, will be aware of various possible variations of at least a supervised machine-learning process 228 that may be used to determine relation between inputs and outputs. Supervised machine-learning processes may include classification algorithms as defined above.

Further referring to FIG. 2, machine learning processes may include at least an unsupervised machine-learning processes 232. An unsupervised machine-learning process, as used herein, is a process that derives inferences in datasets without regard to labels; as a result, an unsupervised machine-learning process may be free to discover any structure, relationship, and/or correlation provided in the data. Unsupervised processes may not require a response variable; unsupervised processes may be used to find interesting patterns and/or inferences between variables, to determine a degree of correlation between two or more variables, or the like.

Still referring to FIG. 2, machine-learning module 200 may be designed and configured to create a machine-learning model 224 using techniques for development of linear regression models. Linear regression models may include ordinary least squares regression, which aims to minimize the square of the difference between predicted outcomes and actual outcomes according to an appropriate norm for measuring such a difference (e.g. a vector-space distance norm); coefficients of the resulting linear equation may be modified to improve minimization. Linear regression models may include ridge regression methods, where the function to be minimized includes the least-squares function plus term multiplying the square of each coefficient by a scalar amount to penalize large coefficients. Linear regression models may include least absolute shrinkage and selection operator (LASSO) models, in which ridge regression is combined with multiplying the least-squares term by a factor of 1 divided by double the number of samples. Linear regression models may include a multi-task lasso model wherein the norm applied in the least-squares term of the LASSO model is the Frobenius norm amounting to the square root of the sum of squares of all terms. Linear regression models may include the elastic net model, a multi-task elastic net model, a least angle regression model, a LARS LASSO model, an orthogonal matching pursuit model, a Bayesian regression model, a logistic regression model, a stochastic gradient descent model, a perceptron model, a passive aggressive algorithm, a robustness regression model, a Huber regression model, or any other suitable model that may occur to persons skilled in the art upon reviewing the entirety of this disclosure. Linear regression models may be generalized in an embodiment to polynomial regression models, whereby a polynomial equation (e.g. a quadratic, cubic or higher-order equation) providing a best predicted output/actual output fit is sought; similar methods to those described above may be applied to minimize error functions, as will be apparent to persons skilled in the art upon reviewing the entirety of this disclosure.

Continuing to refer to FIG. 2, machine-learning algorithms may include, without limitation, linear discriminant analysis. Machine-learning algorithm may include quadratic discriminant analysis. Machine-learning algorithms may include kernel ridge regression. Machine-learning algorithms may include support vector machines, including without limitation support vector classification-based regression processes. Machine-learning algorithms may include stochastic gradient descent algorithms, including classification and regression algorithms based on stochastic gradient descent. Machine-learning algorithms may include nearest neighbors algorithms. Machine-learning algorithms may include various forms of latent space regularization such as variational regularization. Machine-learning algorithms may include Gaussian processes such as Gaussian Process Regression. Machine-learning algorithms may include cross-decomposition algorithms, including partial least squares and/or canonical correlation analysis. Machine-learning algorithms may include naïve Bayes methods. Machine-learning algorithms may include algorithms based on decision trees, such as decision tree classification or regression algorithms. Machine-learning algorithms may include ensemble methods such as bagging meta-estimator, forest of randomized trees, AdaBoost, gradient tree boosting, and/or voting classifier methods. Machine-learning algorithms may include neural net algorithms, including convolutional neural net processes.

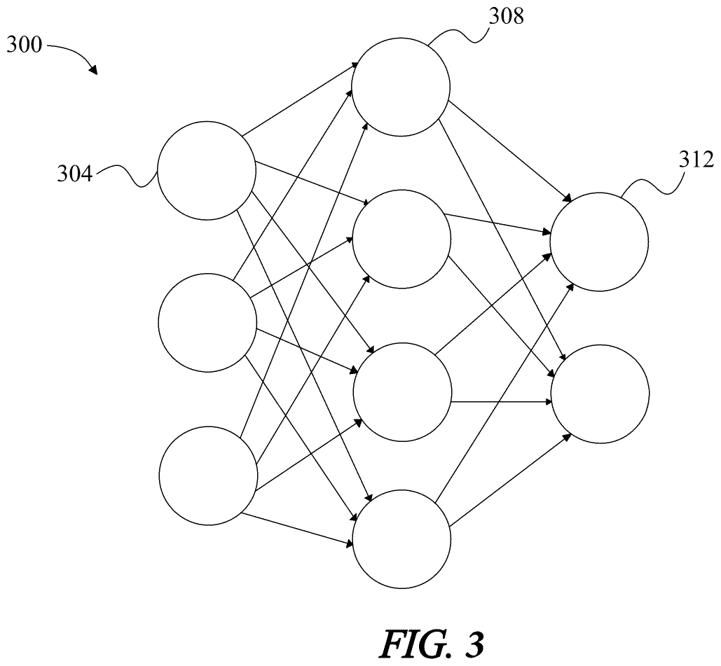

Referring now to FIG. 3, an exemplary embodiment of neural network 300 is illustrated. A neural network 300 also known as an artificial neural network, is a network of “nodes,” or data structures having one or more inputs, one or more outputs, and a function determining outputs based on inputs. Such nodes may be organized in a network, such as without limitation a convolutional neural network, including an input layer of nodes 304, one or more intermediate layers 308, and an output layer of nodes 312. Connections between nodes may be created via the process of “training” the network, in which elements from a training dataset are applied to the input nodes, a suitable training algorithm (such as Levenberg-Marquardt, conjugate gradient, simulated annealing, or other algorithms) is then used to adjust the connections and weights between nodes in adjacent layers of the neural network to produce the desired values at the output nodes. This process is sometimes referred to as deep learning. Connections may run solely from input nodes toward output nodes in a “feed-forward” network, or may feed outputs of one layer back to inputs of the same or a different layer in a “recurrent network.” As a further non-limiting example, a neural network may include a convolutional neural network comprising an input layer of nodes, one or more intermediate layers, and an output layer of nodes. A “convolutional neural network,” as used in this disclosure, is a neural network in which at least one hidden layer is a convolutional layer that convolves inputs to that layer with a subset of inputs known as a “kernel,” along with one or more additional layers such as pooling layers, fully connected layers, and the like.

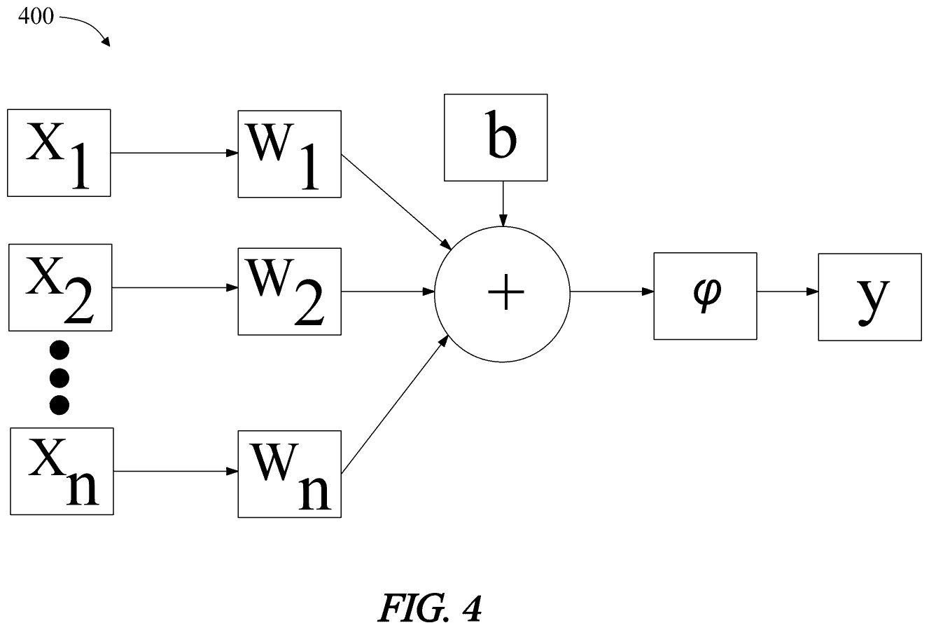

Referring now to FIG. 4, an exemplary embodiment of a node of a neural network is illustrated. A node may include, without limitation a plurality of inputs xᵢ that may receive numerical values from inputs to a neural network containing the node and/or from other nodes. Node may perform a weighted sum of inputs using weights wᵢ that are multiplied by respective inputs xᵢ. Additionally or alternatively, a bias b may be added to the weighted sum of the inputs such that an offset is added to each unit in the neural network layer that is independent of the input to the layer. The weighted sum may then be input into a function φ, which may generate one or more outputs y. Weight wᵢ applied to an input xᵢ may indicate whether the input is “excitatory,” indicating that it has strong influence on the one or more outputs y, for instance by the corresponding weight having a large numerical value, and/or a “inhibitory,” indicating it has a weak effect influence on the one more inputs y, for instance by the corresponding weight.

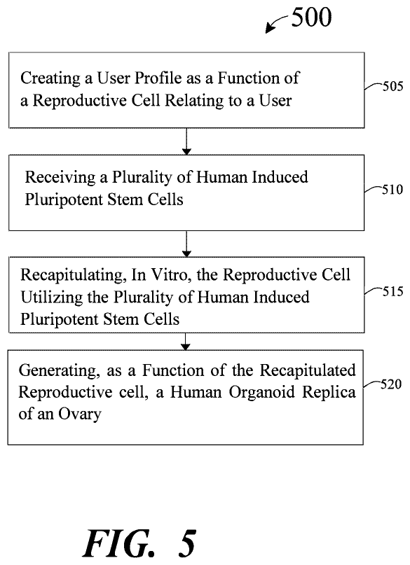

Referring now to FIG. 5, is an exemplary flow diagram of a method for engineering a human organoid replica for reproductive screening. At step 505, method 500 includes creating a user profile as a function of a reproductive cell relating to a user. In some embodiments, creating the user profile includes profiling an ovary at a single cell resolution. This may include receiving an ovarian cell from the user. “Single cell resolution,” as used in this disclosure, is next-generation sequencing technologies used to examine the sequence information from individual cells, providing a higher resolution of cellular differences and a better understanding of the function of an individual cell in the context of its microenvironment. Profiling the ovary may include utilizing in silico target discovery. In some embodiments, creating the user profile may include identifying at least an ovarian cell from an ovary demonstrating a reproductive discrepancy. A “reproductive discrepancy,” as used in this disclosure, is the abnormal cell health or function of a cell in a living organism. For example, a reproductive discrepancy may be epithelial cells dividing uncontrollably and showing signs of tumor growth. The reproductive discrepancy may include a cell health change, metabolic change, expression change, genome change, disease (e.g., endometriosis, polycystic ovary syndrome, and the like) and the like. Reproductive discrepancies may be identified using assays and machine-learning, as described above with reference to FIG. 1. For example, profiling may include high resolution profiling of transcriptome and epigenome at single cell level of ovaries from various aged cadaver donors. Profiling may include analyzing a plurality of ovaries and/or reproductive systems (e.g., fallopian tubes) received from donors of various ages to create a library of epigenetic characteristics. Donor tissue may be analyzed using methods such as oocytes for fertile co-culture, transcriptomics, metabolomics, tissue microscopy and the like. In some embodiments, donor tissue may be compared to the user profile to identity a reproductive discrepancy related to the user. For example, DNA methylation patterns exhibited in a user profile may be compared to donor tissue to compare/contrast and identity age-related diseases such as cancer, osteoarthritis, and neurodegeneration. In some embodiments, comparing a user profile to donor tissue may include utilizing a classifier as described above with reference to FIGS. 1 and 2. For example, the classifier may receive analytical data of an assayed reproductive cell relating to the user, wherein the classifier is configured to output user profile contains at least an identified reproductive discrepancy. The classifier may be trained using training data including correlation between donor tissue, a reproductive knowledge data structure containing information on reproductive health such as disease states, and any other form of data described throughout this disclosure.

At step 510, method 500 includes receiving a plurality of human induced pluripotent stem cells (hiPSCs). hiPSCs may be received from the user and/or donors. At step 515, method 500 includes recapitulating, in vitro, the ovarian cell utilizing the plurality of human induced pluripotent stem cells. In some embodiments, recapitulating the ovarian cell may include replicating an ovarian cell demonstrating a reproductive discrepancy. Recapitulating the ovarian cell may include producing engineered reproductive cells as described above with reference to FIG. 1. Recapitulating the ovarian cell further may further include utilizing transcription factor-directed cell differentiation as described above with reference to FIG. 1.

At step 520, method 500 includes generating, as a function of the recapitulated ovarian cell, a human organoid replica of an ovary as described and with reference to FIG. 1. Generating the human organoid replica may include generating a human organoid replica containing a reproductive cell, such as an ovarian cell, demonstrating a reproductive discrepancy. In some embodiments, generating the human organoid replica may include utilizing bioprinting.

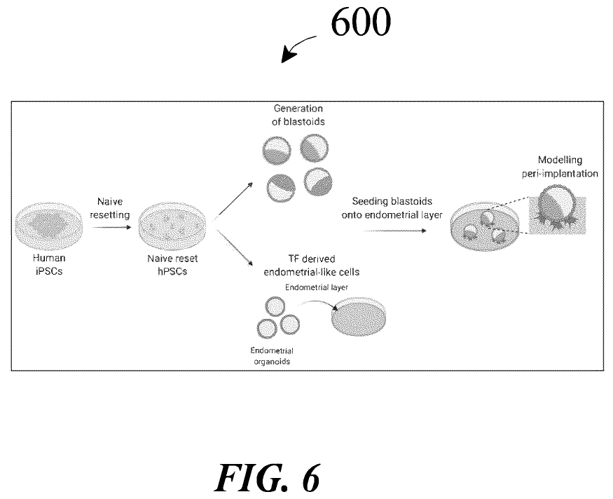

Referring now to FIG. 6, is an exemplary diagram of a human organoid replica applied as a disease model. The human organoid replica may be used in developing tools for improvement in embryo culture and implantation. During implantation, a symphony of interaction between the trophoblast originated from the trophectoderm of the implanting blastocyst and the endometrium may lead to a successful pregnancy. Defective interaction between the trophoblast and endometrium may result in implantation failure, pregnancy loss, and a number of pregnancy complications. In forming the disease model, Human iPSCs may be cultured in vitro. In some embodiments, Human iPSCs may be reprogrammed to naïve-like state. “Naïve human pluripotent stem cells (hPSC),” as used in this disclosure, are cells in vitro resembling the inner cell mass of human embryonic day (E) 6-7 preimplantation blastocysts. Compared to a primed state, cells in the naive pluripotent state may be more amenable to genome editing, present higher proliferative rate, and have higher chimeric integration potential. Additionally, naïve human pluripotent stem cells may resemble the embryonic epiblast at an earlier time-point in development than conventional, ‘primed’ hPSC. By exposing naive human embryonic stem cells to a cellular differentiation media, they may differentiate into the embryonic and the extraembryonic cell lineages, including the SOX2 positive epiblast-like cells, GATA6 positive hypoblast-like cells and GATA3 positive trophoblast-like cells. Human blastoids may be generated be generated from human embryonic stem cells. A “blastoid,” as used in this disclosure, is a stem cell-based embryo model which, morphologically and transcriptionally resembles the early, pre-implantation, mammalian conceptus, called the blastocyst.

Still referring to FIG. 6, Naïve human pluripotent stem cells may be used to generate transcription factor derived endometrial-like cells of an endometrial layer (i.e., endometrial organoids) using methods as described above with reference to at least FIG. 1. The endometrium, as used herein, is the innermost lining layer of the uterus, and functions to prevent adhesions between the opposed walls of the myometrium, thereby maintaining the patency of the uterine cavity. During the menstrual cycle or estrous cycle, the endometrium grows to a thick, blood vessel-rich, glandular tissue layer. This represents an optimal environment for the implantation of a blastocyst upon its arrival in the uterus. “Implantation,” as used in this disclosure, is the process that leads from blastocyst attachment to its embedding in the uterine wall. Failure of implantation may be linked to pregnancy loss. Toxic agents can interfere directly with the process of implantation and therefore may account for unexplained implantation failures. It may be difficult to gain a better understanding of the events in human pregnancy that occur during and just after implantation because such pregnancies may not yet be clinically detectable. Animal models of human placentation may be inadequate. In vitro models that utilize immortalized cell lines and cells derived from trophoblast cancers have multiple limitations. Primary cell and tissue cultures often have limited lifespans and cannot be obtained from the peri-implantation period. The human blastoids derived as described above may be seeded on the blastoids onto the endometrial organoid for peri-implantation modeling. Additionally, this may be used for disease modeling to discover toxins and other agents that contribute to pregnancy loss and implantation failure.

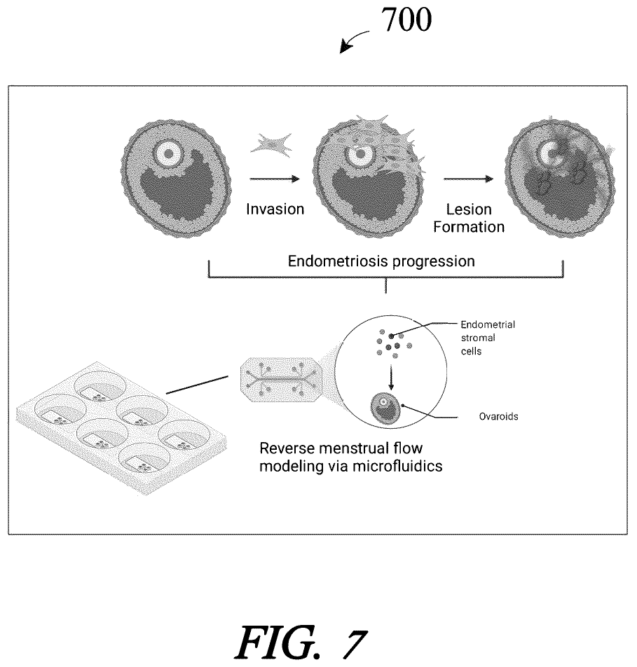

Referring now to FIG. 7, is an exemplary diagram of a human organoid replica applied as a druggable disease model. Human organoid replica may be generated to model human endometriosis. “Endometriosis,” as used in this disclosure, is a disease of the female reproductive system in which cells similar to those in the endometrium, the layer of tissue that normally covers the inside of the uterus, grow outside the uterus. Most often this is on the ovaries, fallopian tubes, and tissue around the uterus and ovaries; in rare cases it may also occur in other parts of the body. The cause is not entirely clear. Risk factors may include having a family history of the condition. The areas of endometriosis bleed each month (menstrual period), resulting in inflammation and scarring. In some embodiments, human organoid replica may be generated to model various stages of endometriosis progression such as invasion and lesion formation. Endometrial stromal cells may be received from a donor and cultured in vitro to derive an ovaroid (ovarian organoid). “Endometrial stromal cells,” as used in this disclosure, are the main supportive (stromal) cell type that underlies endometrial surface epithelium and surrounds glands. In the human endometrium, stromal cells may mediate the proliferative response of epithelial cells to the steroid hormones' estrogen and progesterone.

Still referring to FIG. 7, human organoid replica may be configured to model retrograde menstruation utilizing microfluidics as described above. “Retrograde menstruation,” as used in this disclosure, is the inverse flow of menstrual fluid which leaves the uterus through the fallopian tubes into the pelvic cavity. The menstrual fluid present in pelvic cavity because of the retrograde menstruation may contain the blood cells (erythrocytes) and endometrial tissue. The role of blood cells present in pelvic cavity can be considered as a complementary factor for development of endometriosis alongside the endometrial cells. Specifically, increased degradation of blood cells and insufficient inactivation of hemoglobin in pelvic cavity may be crucial factor for the disease development. In an embodiment, human organoid replica may be configured to model any disease state and/or medical condition.

It is to be noted that any one or more of the aspects and embodiments described herein may be conveniently implemented using one or more machines (e.g., one or more computing devices that are utilized as a user computing device for an electronic document, one or more server devices, such as a document server, etc.) programmed according to the teachings of the present specification, as will be apparent to those of ordinary skill in the computer art. Appropriate software coding can readily be prepared by skilled programmers based on the teachings of the present disclosure, as will be apparent to those of ordinary skill in the software art. Aspects and implementations discussed above employing software and/or software modules may also include appropriate hardware for assisting in the implementation of the machine executable instructions of the software and/or software module.

Such software may be a computer program product that employs a machine-readable storage medium. A machine-readable storage medium may be any medium that is capable of storing and/or encoding a sequence of instructions for execution by a machine (e.g., a computing device) and that causes the machine to perform any one of the methodologies and/or embodiments described herein. Examples of a machine-readable storage medium include, but are not limited to, a magnetic disk, an optical disc (e.g., CD, CD-R, DVD, DVD-R, etc.), a magneto-optical disk, a read-only memory “ROM” device, a random access memory “RAM” device, a magnetic card, an optical card, a solid-state memory device, an EPROM, an EEPROM, and any combinations thereof. A machine-readable medium, as used herein, is intended to include a single medium as well as a collection of physically separate media, such as, for example, a collection of compact discs or one or more hard disk drives in combination with a computer memory. As used herein, a machine-readable storage medium does not include transitory forms of signal transmission.

Such software may also include information (e.g., data) carried as a data signal on a data carrier, such as a carrier wave. For example, machine-executable information may be included as a data-carrying signal embodied in a data carrier in which the signal encodes a sequence of instruction, or portion thereof, for execution by a machine (e.g., a computing device) and any related information (e.g., data structures and data) that causes the machine to perform any one of the methodologies and/or embodiments described herein.

Examples of a computing device include, but are not limited to, an electronic book reading device, a computer workstation, a terminal computer, a server computer, a handheld device (e.g., a tablet computer, a smartphone, etc.), a web appliance, a network router, a network switch, a network bridge, any machine capable of executing a sequence of instructions that specify an action to be taken by that machine, and any combinations thereof. In one example, a computing device may include and/or be included in a kiosk.

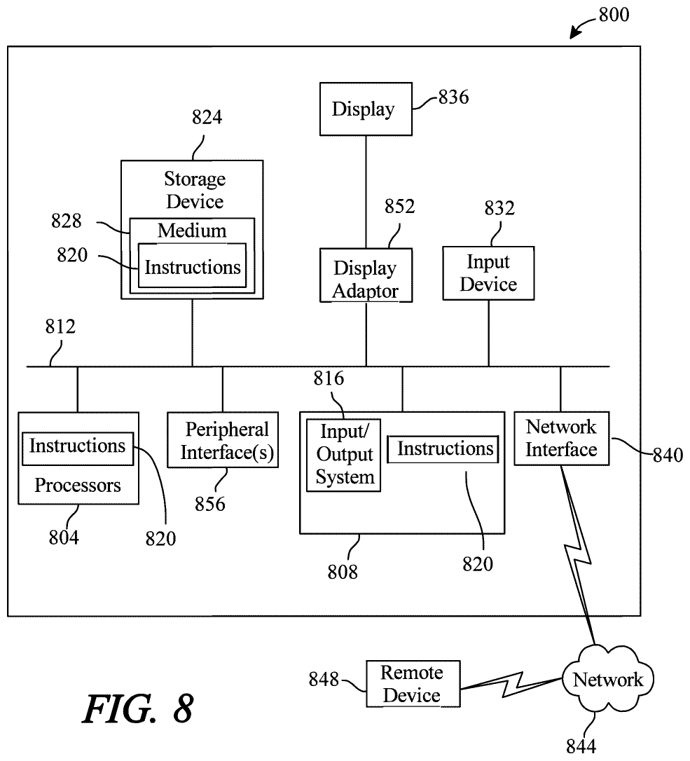

FIG. 8 shows a diagrammatic representation of one embodiment of a computing device in the exemplary form of a computer system 800 within which a set of instructions for causing a control system to perform any one or more of the aspects and/or methodologies of the present disclosure may be executed. It is also contemplated that multiple computing devices may be utilized to implement a specially configured set of instructions for causing one or more of the devices to perform any one or more of the aspects and/or methodologies of the present disclosure. Computer system 800 includes a processor 804 and a memory 808 that communicate with each other, and with other components, via a bus 812. Bus 812 may include any of several types of bus structures including, but not limited to, a memory bus, a memory controller, a peripheral bus, a local bus, and any combinations thereof, using any of a variety of bus architectures.

Processor 804 may include any suitable processor, such as without limitation a processor incorporating logical circuitry for performing arithmetic and logical operations, such as an arithmetic and logic unit (ALU), which may be regulated with a state machine and directed by operational inputs from memory and/or sensors; processor 804 may be organized according to Von Neumann and/or Harvard architecture as a non-limiting example. Processor 804 may include, incorporate, and/or be incorporated in, without limitation, a microcontroller, microprocessor, digital signal processor (DSP), Field Programmable Gate Array (FPGA), Complex Programmable Logic Device (CPLD), Graphical Processing Unit (GPU), general purpose GPU, Tensor Processing Unit (TPU), analog or mixed signal processor, Trusted Platform Module (TPM), a floating point unit (FPU), and/or system on a chip (SoC).

Memory 808 may include various components (e.g., machine-readable media) including, but not limited to, a random-access memory component, a read only component, and any combinations thereof. In one example, a basic input/output system 816 (BIOS), including basic routines that help to transfer information between elements within computer system 800, such as during start-up, may be stored in memory 808. Memory 808 may also include (e.g., stored on one or more machine-readable media) instructions (e.g., software) 820 embodying any one or more of the aspects and/or methodologies of the present disclosure. In another example, memory 808 may further include any number of program modules including, but not limited to, an operating system, one or more application programs, other program modules, program data, and any combinations thereof.

Computer system 800 may also include a storage device 824. Examples of a storage device (e.g., storage device 824) include, but are not limited to, a hard disk drive, a magnetic disk drive, an optical disc drive in combination with an optical medium, a solid-state memory device, and any combinations thereof. Storage device 824 may be connected to bus 812 by an appropriate interface (not shown). Example interfaces include, but are not limited to, SCSI, advanced technology attachment (ATA), serial ATA, universal serial bus (USB), IEEE 1394 (FIREWIRE), and any combinations thereof. In one example, storage device 824 (or one or more components thereof) may be removably interfaced with computer system 800 (e.g., via an external port connector (not shown)). Particularly, storage device 824 and an associated machine-readable medium 828 may provide nonvolatile and/or volatile storage of machine-readable instructions, data structures, program modules, and/or other data for computer system 800. In one example, software 820 may reside, completely or partially, within machine-readable medium 828. In another example, software 820 may reside, completely or partially, within processor 804.

Computer system 800 may also include an input device 832. In one example, a user of computer system 800 may enter commands and/or other information into computer system 800 via input device 832. Examples of an input device 832 include, but are not limited to, an alpha-numeric input device (e.g., a keyboard), a pointing device, a joystick, a gamepad, an audio input device (e.g., a microphone, a voice response system, etc.), a cursor control device (e.g., a mouse), a touchpad, an optical scanner, a video capture device (e.g., a still camera, a video camera), a touchscreen, and any combinations thereof. Input device 832 may be interfaced to bus 812 via any of a variety of interfaces (not shown) including, but not limited to, a serial interface, a parallel interface, a game port, a USB interface, a FIREWIRE interface, a direct interface to bus 812, and any combinations thereof. Input device 832 may include a touch screen interface that may be a part of or separate from display 836, discussed further below. Input device 832 may be utilized as a user selection device for selecting one or more graphical representations in a graphical interface as described above.

A user may also input commands and/or other information to computer system 800 via storage device 824 (e.g., a removable disk drive, a flash drive, etc.) and/or network interface device 840. A network interface device, such as network interface device 840, may be utilized for connecting computer system 800 to one or more of a variety of networks, such as network 844, and one or more remote devices 848 connected thereto. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network, such as network 844, may employ a wired and/or a wireless mode of communication. In general, any network topology may be used. Information (e.g., data, software 820, etc.) may be communicated to and/or from computer system 800 via network interface device 840.

Computer system 800 may further include a video display adapter 852 for communicating a displayable image to a display device, such as display device 836. Examples of a display device include, but are not limited to, a liquid crystal display (LCD), a cathode ray tube (CRT), a plasma display, a light emitting diode (LED) display, and any combinations thereof. Display adapter 852 and display device 836 may be utilized in combination with processor 804 to provide graphical representations of aspects of the present disclosure. In addition to a display device, computer system 800 may include one or more other peripheral output devices including, but not limited to, an audio speaker, a printer, and any combinations thereof. Such peripheral output devices may be connected to bus 812 via a peripheral interface 856. Examples of a peripheral interface include, but are not limited to, a serial port, a USB connection, a FIREWIRE connection, a parallel connection, and any combinations thereof.

The foregoing has been a detailed description of illustrative embodiments of the invention. Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments, what has been described herein is merely illustrative of the application of the principles of the present invention. Additionally, although particular methods herein may be illustrated and/or described as being performed in a specific order, the ordering is highly variable within ordinary skill to achieve apparatuses, methods, systems, and software according to the present disclosure. Accordingly, this description is meant to be taken only by way of example, and not to otherwise limit the scope of this invention.

Exemplary embodiments have been disclosed above and illustrated in the accompanying drawings. It will be understood by those skilled in the art that various changes, omissions and additions may be made to that which is specifically disclosed herein without departing from the spirit and scope of the present invention.

What is claimed is:

1. A platform for engineering a human organoid replica for reproductive screening, the platform comprising:
an engineered reproductive cell;
a detector that measures a response by the engineered reproductive cell upon exposure of the engineered reproductive cell to a stimulus;
a computing device in communication with the detector wherein the computing further comprises:
at least a processor; and
a memory communicatively connected to the processor, the memory containing instructions configuring the at least a processor to:
process stimulus data received from the detector; and
compare the stimulus data to a user profile.

2. The platform of claim 1, wherein the engineered reproductive cell is generated utilizing bioprinting.

3. The platform of claim 1, wherein the stimulus relates to drug metabolism.

4. The platform of claim 1, wherein the stimulus relates to a toxicity screening.

5. The platform of claim 1, wherein the stimulus relates to a disease model.

6. The platform of claim 1, wherein the stimulus relates to an epigenetic model.

7. The platform of claim 1, wherein processing the stimulus data comprises utilizing cell viability assays.

8. The platform of claim 1, wherein processing the stimulus data comprises utilizing a classifier configured to generate an image-based profile assessment.

9. The platform of claim 1, wherein comparing the stimulus data to a user profile comprises generating a reproductive discrepancy treatment plan.

10. The platform of claim 1, further comprising utilizing the engineered human organoid replica for embryo culture peri-implantation modeling.

11. A method for engineering a human organoid replica for reproductive screening, the method comprising:
creating a user profile as a function of a reproductive cell relating to a user;
receiving a plurality of human induced pluripotent stem cells;
recapitulating, in vitro, the reproductive cell utilizing the plurality of human induced pluripotent stem cells; and
generating, as a function of the recapitulated reproductive cell, a human organoid replica of an ovary.

12. The method of claim 11, wherein creating the user profile further comprises profiling an ovary at a single cell resolution.

13. The method of claim 12, wherein profiling the ovary further comprises utilizing in silico target discovery.

14. The method of claim 11, wherein creating the user profile further comprises identifying at least a reproductive cell from an ovary, wherein the at least a reproductive cell demonstrates a reproductive discrepancy.

15. The method of claim 14, wherein the reproductive discrepancy comprises a cell health change.

16. The method of claim 14, wherein the reproductive discrepancy comprises endometriosis.

17. The method of claim 11, wherein recapitulating the reproductive cell further comprises replicating a reproductive cell demonstrating a reproductive discrepancy.

18. The method of claim 11, wherein recapitulating the reproductive cell further comprises utilizing transcription factor-directed cell differentiation.

19. The method of claim 11, wherein generating the human organoid replica further comprises generating a human organoid replica containing a reproductive cell demonstrating a reproductive discrepancy.

20. The method of claim 11, wherein generating the human organoid replica further comprises utilizing bioprinting.
